(19) United States
(12) Patent Application Publication
(10) Pub. No.: US 2014/0039586 A1
(43) Pub. Date: Feb. 6, 2014

(54) SYSTEMS AND METHODS FOR MAKING AND USING A MULTI-LEAD INTRODUCER FOR USE WITH ELECTRICAL STIMULATION SYSTEMS

(71) Applicant: BOSTON SCIENTIFIC NEUROMODULATION CORPORATION, Valencia, CA (US)

(72) Inventors: John Michael Barker, Ventura, CA (US); Jacob B. Leven, Huntington Beach, CA (US); Michelle Deng, Chanhassen, MN (US)

(73) Assignee: BOSTON SCIENTIFIC NEUROMODULATION CORPORATION, Valencia, CA (US)

(21) Appl. No.: 13/951,024

(22) Filed: Jul. 25, 2013

Related U.S. Application Data
(60) Provisional application No. 61/679,620, filed on Aug. 3, 2012.

(51) Int. Cl. A61N 1/05

(52) U.S. Cl. CPC A61N 1/0551
USPC 607/116

(57) ABSTRACT

A multi-lead introducer for facilitating implantation of stimulation leads includes a needle assembly for concurrently implanting multiple leads into a patient. The needle assembly includes a first needle with a first needle lumen configured to receive a first lead. A first sharpened tip is disposed along a first end of the first needle for piercing patient tissue. A second needle defines a second needle lumen configured to receive a second lead. A second sharpened tip is disposed along a first end of the second needle for piercing patient tissue. A hub is coupled to second ends of both the first needle and the second needle. The hub defines a hub lumen that is in communication with both the first needle lumen and the second needle lumen. The hub lumen is configured to concurrently receive both the first lead and the second lead.

### CROSS-REFERENCE TO RELATED APPLICATIONS

This application claims the benefit under 35 U.S.C. §119(e) of U.S. Provisional Patent Application Ser. No. 61/679,620 filed on Aug. 3, 2012, which is incorporated herein by reference.

### FIELD

The present invention is directed to the area of implantable electrical stimulation systems and methods of making and using the systems. The present invention is also directed to introducer needles configured and arranged for concurrently implanting multiple electrical stimulation leads into a patient, as well as methods of making and using the introducer needles, leads, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a multi-lead introducer for facilitating implantation of at least one electrical stimulation lead includes a needle assembly configured and arranged to concurrently implant a plurality of leads into a patient. The needle assembly includes a first needle having a first end, an opposing second end, and a longitudinal length. The first needle defines a first needle lumen extending along the longitudinal length of the first needle from the first end to the second end. The first needle lumen is configured and arranged to receive a first lead of the plurality of leads. A first sharpened tip is disposed along the first end of the first needle. The first sharpened tip is configured and arranged to pierce patient tissue. A second needle has a first end, an opposing second end, and a longitudinal length. The second needle defines a second needle lumen extending along the longitudinal length of the second needle from the first end to the second end. The second needle lumen is configured and arranged to receive a second lead of the plurality of leads. A second sharpened tip is disposed along the first end of the second needle. The second sharpened tip is configured and arranged to pierce patient tissue. A hub is coupled to both the second end of the first needle and the second end of the second needle. The hub defines at least one hub lumen that is in communication with both the first needle lumen and the second needle lumen. The at least one hub lumen is configured and arranged to concurrently receive both the first lead and the second lead.

In another embodiment, a multi-lead introducer for facilitating implantation of at least one electrical stimulation lead includes a needle assembly configured and arranged to concurrently implant a plurality of leads into a patient. The needle assembly includes a single needle having a first end, an opposing second end, and a longitudinal length. The single needle defines a single needle lumen extending along the longitudinal length of the single needle from the first end to the opposing second end. The single needle lumen is configured and arranged to concurrently receive both a first lead and a second lead of the plurality of leads. A sharpened tip is disposed along the first end of the single needle. The sharpened tip is configured and arranged to pierce patient tissue. A hub is coupled to the second end of the single needle. The hub defines at least one hub lumen that is in communication with the single needle lumen. The at least one hub lumen is configured and arranged to concurrently receive both the first lead and the second lead.

In yet another embodiment, a multi-lead introducer for facilitating implantation of at least one electrical stimulation lead includes a needle assembly configured and arranged to concurrently implant a plurality of leads into a patient. The needle assembly includes a single needle having a first end, an opposing second end, and a longitudinal length. The single needle defines a first needle lumen and a second needle lumen. Each of the first needle lumen and the second needle lumen extends along the longitudinal length of the single needle from the first end to the second end. The first needle lumen is configured and arranged to receive a first lead the plurality of leads. The second needle lumen is configured and arranged to receive a second lead the plurality of leads. A sharpened tip is disposed along the first end of the single needle. The sharpened tip is configured and arranged to pierce patient tissue. A hub is coupled to the second end of the single needle. The hub defines at least one hub lumen that is in communication with each of the first needle lumen and the second needle lumen. The at least one hub lumen is configured and arranged to concurrently receive both the first lead and the second lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2A is a schematic view of one embodiment of a proximal portion of a lead and a control module of an electrical stimulation system, according to the invention;

FIG. 2B is a schematic view of one embodiment of a proximal portion of a lead and a lead extension of an electrical stimulation system, according to the invention;

FIG. 3A is a schematic perspective view of one embodiment of a multi-lead introducer having a needle assembly that includes a plurality of needles coupled to a common hub, the multi-lead introducer configured and arranged for concurrently inserting multiple leads into a patient, according to the invention;

FIG. 3B is a schematic perspective exploded view of one embodiment of the multi-lead introducer of FIG. 3A, according to the invention;

FIG. 3C is a schematic perspective, close-up view of one embodiment of portions of stylets disposed in a distal end of the needle assembly of FIG. 3A, according to the invention;

FIG. 4A is a schematic perspective view of one embodiment of a needle coupler disposed over a distal portion of the needle assembly of FIG. 3A, according to the invention;

FIG. 4B is a schematic perspective view of one embodiment of the needle coupler of FIG. 4A disposed over a proximal portion of the needle assembly of FIG. 3A, according to the invention;

FIG. 4C is a schematic close-up, perspective view of one embodiment of the needle coupler of FIG. 4A disposed over a distal portion of the needle assembly of JIG. 3A, according to the invention;

FIG. 5A is a schematic perspective, close-up view of one embodiment of a distal tip insert coupled to the distal ends of the needles of the needle assembly of FIG. 3A, according to the invention;

FIG. 5B is a schematic perspective view of one embodiment of the distal tip insert of FIG. 5A, according to the invention;

FIG. 6A is a schematic perspective view of another embodiment of a multi-lead introducer, the multi-lead introducer having a needle assembly that includes a single needle coupled to a hub, the needle defining multiple lumens configured and arranged for concurrently inserting multiple leads into a patient, according to the invention;

FIG. 6B is a schematic perspective, close-up view of one embodiment of portions of stylets disposed in a distal end of the needle assembly of FIG. 6A, according to the invention;

FIG. 7A is a schematic perspective view of yet another embodiment of a multi-lead introducer, the multi-lead introducer having a needle assembly that includes a single needle coupled to a hub, the needle defining a single lumen configured and arranged for concurrently inserting multiple leads into a patient, according to the invention;

FIG. 7B is a schematic perspective, exploded view of one embodiment of the multi-lead introducer of FIG. 7A, according to the invention;

FIG. 7C is a schematic transverse cross-sectional view of one embodiment of the single lumen of the single needle of FIG. 7A, according to the invention;

FIG. 7D is a schematic perspective, close-up view of one embodiment of portions of a stylet disposed in a distal end of the needle assembly of FIG. 7A, according to the invention;

FIG. 8A is a schematic perspective view of one embodiment of the multi-lead introducer of FIG. 7A, the multi-lead introducer including a needle assembly having a needle, a stylet configured for insertion into the needle, and a divider also configured for insertion into the needle, according to the invention;

FIG. 8B is a schematic transverse cross-sectional view of one embodiment of the needle of FIG. 8A, according to the invention;

FIG. 8C is a schematic transverse cross-sectional view of one embodiment of the divider of FIG. 8A, according to the invention;

FIG. 8D is a schematic transverse cross-sectional view of one embodiment of the stylet of FIG. 8A, according to the invention;

FIG. 8E is a schematic perspective view of one embodiment of the divider of FIG. 8A disposed in the needle lumen of FIG. 8A; and a transverse cross-sectional view of one embodiment of two leads disposed in the needle lumen, with the two leads separated from one another by the divider, where the needle lumen defines apertures and the divider includes protrusions that mate with the apertures when the divider is disposed in the needle lumen, according to the invention;

FIG. 8F is a schematic perspective view of one embodiment of the multi-lead introducer of FIG. 7A, the multi-lead introducer including a needle assembly having a needle coupled to a huh and a stylet configured for insertion into the needle, according to the invention;

FIG. 8G is a schematic transverse cross-sectional view of one embodiment of the hub of FIG. 8F, according to the invention;

FIG. 8H is a schematic transverse cross-sectional view of one embodiment of the needle of FIG. 8F, according to the invention;

FIG. 8I is a schematic transverse cross-sectional view of one embodiment of the divider of FIG. 8F, according to the invention;

FIG. 8J is a schematic perspective view of another embodiment of the multi-lead introducer of FIG. 7A, the multi-lead introducer including a needle assembly having a needle coupled to a hub, a stylet configured for insertion into the needle and hub, and a divider configured for insertion into the needle and hub, where the divider includes a tab that mates with a notch defined in the huh, according to the invention;

FIG. 9 is a schematic perspective view of one embodiment of a bend disposed along the distal end of the needle assembly of FIG. 7A, according to the invention; and

FIG. 10 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to introducer needles configured and arranged for concurrently implanting multiple electrical stimulation leads into a patient, as well as methods of making and using the introducer needles, leads, and electrical stimulation systems.

Suitable implantable electrical stimulation systems include, but are not limited to, a least one lead with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,672,734; 7,761,165; 7,949,395; 7,974,706; 8,175,710; 8,224,450; and 8,364,278; and U.S. Patent Application Publication No. 2007/0150036, all of which are incorporated by reference.

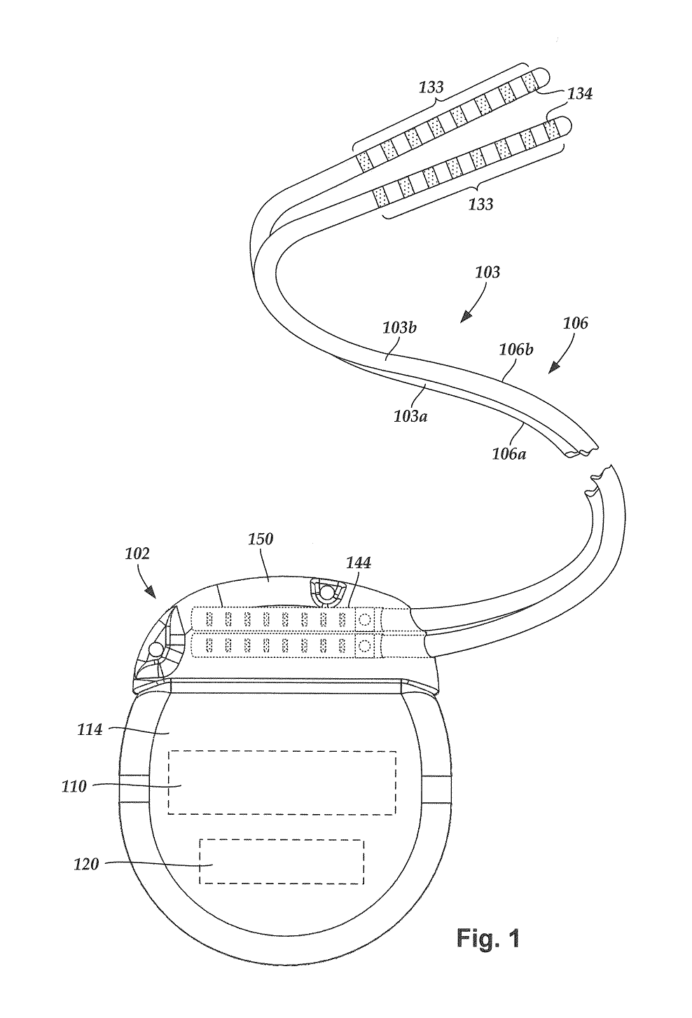

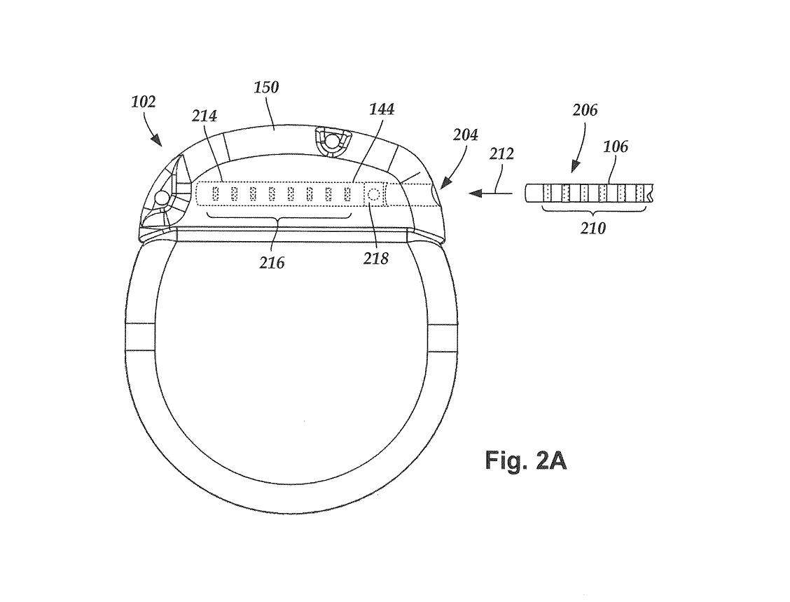

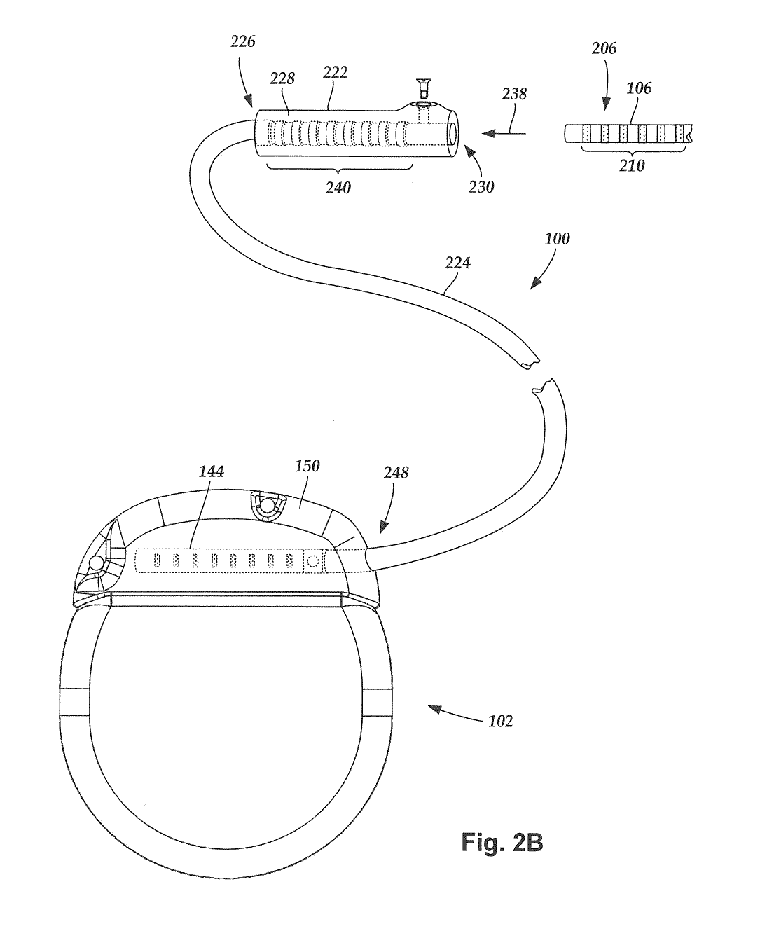

FIG. 1 illustrates schematically one embodiment of an electrical stimulation system 100. The electrical stimulation system includes a control module (e.g., a stimulator or pulse generator) 102 and multiple leads 103 coupled to the control module 102. The leads 103 each include one or more lead bodies 106, where each lead body includes an array of electrodes 133, such as electrode 134, and terminals (e.g., 210 in FIGS. 2A and 236 of FIG. 2B). In FIG. 1, two leads 103a and 103b are shown, where the lead 103a includes the lead body 106a and the lead 103b includes the lead body 106b.

The control module 102 typically includes an electronic subassembly 110 and an optional power source 120 disposed in a sealed housing 114. The control module 102 typically includes a connector 144 (FIG. 2A, see also 222 and 250 of FIG. 2B) into which the proximal end of the one or more lead bodies 106 can be plugged to make an electrical connection via conductive contacts on the control module 102 and terminals on each of the one or more leads 103. In at least some embodiments, a lead is isodiametric along a longitudinal length of the lead body 106. In addition, one or more lead extensions 224 (see FIG. 2B) can be disposed between the one or more lead bodies 106 and the control module 102 to extend the distance between the one or more lead bodies 106 and the control module 102 of the embodiment shown in FIG. 1.

The electrical stimulation system or components of the electrical stimulation system, including one or more of the lead bodies 106 and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. The number of electrodes 134 in each array 133 may vary. For example, there can be two, four, six, eight, ten, twelve, fourteen, sixteen, or more electrodes 134. As will be recognized, other numbers of electrodes 134 may also be used.

The electrodes of one or more lead bodies 106 are typically disposed in, or separated by, a non-conductive, biocompatible material such as, for example, silicone, polyurethane, polyetheretherketone (“PEEK”), epoxy, and the like or combinations thereof. The lead bodies 106 may be formed in the desired shape by any process including, for example, molding (including injection molding), casting, and the like. The non-conductive material typically extends from the distal end of the one or more lead bodies 106 to the proximal end of each of the one or more lead bodies 106.

Terminals (e.g., 210 in FIGS. 2A and 236 of FIG. 2B) are typically disposed at the proximal end of the one or more lead bodies 106 of the electrical stimulation system 100 for connection to corresponding conductive contacts (e.g., 214 in FIGS. 2A and 240 of FIG. 2B) in connectors (e.g., 144 in FIGS. 1-2A and 222 and 250 of FIG. 2B) disposed on, for example, the control module 102 (or to conductive contacts on a lead extension, an operating room cable, or an adaptor). Conductor wires (not shown) extend from the terminals (e.g., 210 in FIGS. 2A and 236 of FIG. 2B) to the electrodes 134. Typically, one or more electrodes 134 are electrically coupled to a terminal (e.g., 210 in FIGS. 2A and 236 of FIG. 2B). In at least some embodiments, each terminal (e.g., 210 in FIGS. 2A and 236 of FIG. 2B) is only connected to one electrode 134.

The conductor wires may be embedded in the non-conductive material of the lead body 106 or can be disposed in one or more lumens (not shown) extending along the lead body 106. In some embodiments, there is an individual lumen for each conductor wire. In other embodiments, two or more conductor wires may extend through a lumen. There may also be one or more lumens (not shown) that open at, or near, the proximal end of the lead body 106, for example, for inserting a stylet wire to facilitate placement of the lead body 106 within a body of a patient. Additionally, there may also be one or more lumens (not shown) that open at, or near, the distal end of the lead body 106, for example, for infusion of drugs or medication into the site of implantation of the one or more lead bodies 106. In at least one embodiment, the one or more lumens may be flushed continually, or on a regular basis, with saline, epidural fluid, or the like. In at least some embodiments, the one or more lumens can be permanently or removably sealable at the distal end.

In at least some embodiments, leads are coupled to connectors disposed on control modules. In FIG. 2A, a lead 208 is shown configured and arranged for insertion to the control module 102. The connector 144 includes a connector housing 202. The connector housing 202 defines at least one port 204 into which a proximal end 206 of a lead 208 with terminals 210 can be inserted, as shown by directional arrow 212. The connector housing 202 also includes a plurality of conductive contacts 214 for each port 204. When the lead 208 is inserted into the port 204, the conductive contacts 214 can be aligned with the terminals 210 on the lead 208 to electrically couple the control module 102 to the electrodes (134 of FIG. 1) disposed at a distal end of the lead 208. Examples of connectors in control modules are found in, for example, U.S. Pat. Nos. 7,244,150 and 8,224,450, which are incorporated by reference.

In FIG. 2B, a connector 222 is disposed on a lead extension 224. The connector 222 is shown disposed at a distal end 226 of the lead extension 224. The connector 222 includes a connector housing 228. The connector housing 228 defines at least one port 230 into which a proximal end 232 of a lead 234 with terminals 236 can be inserted, as shown by directional arrow 238. The connector housing 228 also includes a plurality of conductive contacts 240. When the lead 234 is inserted into the port 230, the conductive contacts 240 disposed in the connector housing 228 can be aligned with the terminals 236 on the lead 234 to electrically couple the lead extension 224 to the electrodes (134 of FIG. 1) disposed at a distal end (not shown) of the lead 234.

In at least some embodiments, the proximal end of a lead extension is similarly configured and arranged as a proximal end of a lead. The lead extension 224 may include a plurality of conductive wires (not shown) that electrically couple the conductive contacts 240 to a proximal end 248 of the lead extension 224 that is opposite to the distal end 226. In at least some embodiments, the conductive wires disposed in the lead extension 224 can be electrically coupled to a plurality of terminals (not shown) disposed on the proximal end 248 of the lead extension 224. In at least some embodiments, the proximal end 248 of the lead extension 224 is configured and arranged for insertion into a connector disposed in another lead extension. In other embodiments, the proximal end 248 of the lead extension 224 is configured and arranged for insertion into a connector disposed in a control module. As an example, in FIG. 2B the proximal end 248 of the lead extension 224 is inserted into a connector 250 disposed in a control module 252.

In at least some situations, it may be advantageous to implant two or more leads into the patient to expand stimulation coverage from what might be achievable using a single lead. For example, in at least some instances it may be desirable to stimulate a patient along two or more regions in proximity to one another, or along a single region that is larger than might be stimulated using a single lead.

In the case of implantation of multiple percutaneous leads, when a conventional lead introducer needle is used to individually implant each of the multiple leads patient skin may be separately pierced by the introducer needle during each individual lead implantation, thereby potentially causing extended pain and anxiety for the patient. Additionally, once each of the individual leads is inserted into the patient, significant time and effort may be spent aligning the leads with one another into a desired stimulation arrangement (e.g., side-by-side, end-to-end, or the like).

As herein described, a multi-lead introducer enables concurrent implantation of multiple percutaneous leads into the patient using a single lead introducer. In at least some embodiments, the multi-lead introducer enables implantation of multiple percutaneous leads using only a single patient skin pierce. In at least some embodiments, the multi-lead introducer enables implantation of two or more leads, where each of the two or more leads is aligned side-by-side with one another when the leads are inserted. In which case, post-insertion alignment of the leads may be easier when the leads are implanted using the multi-lead introducer than when the leads are individually implanted using a conventional introducer needle.

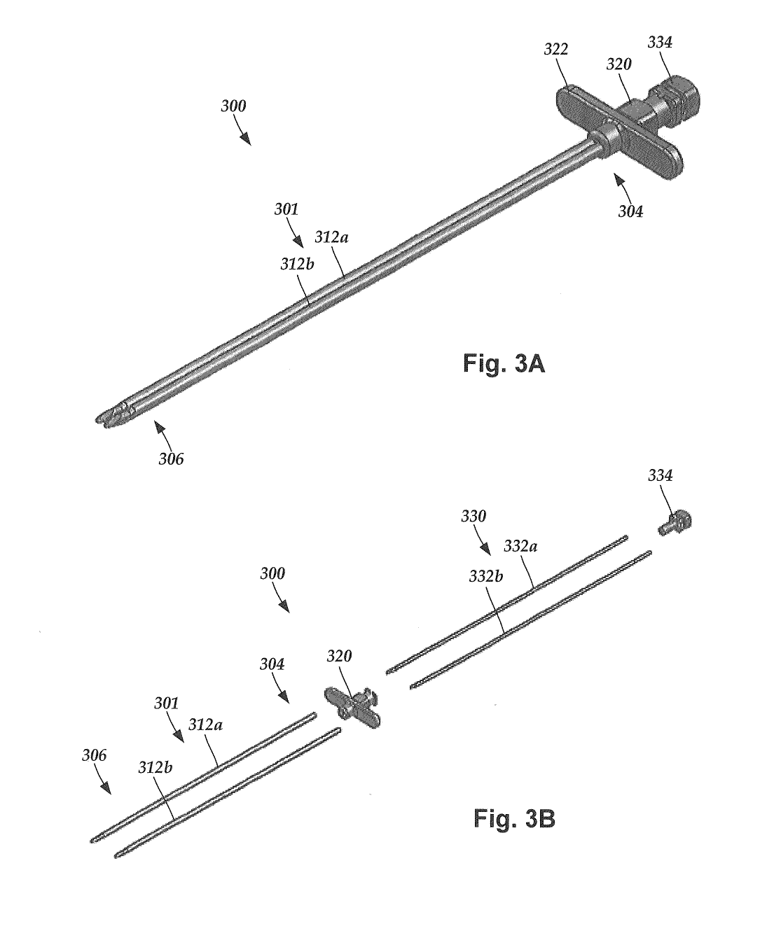

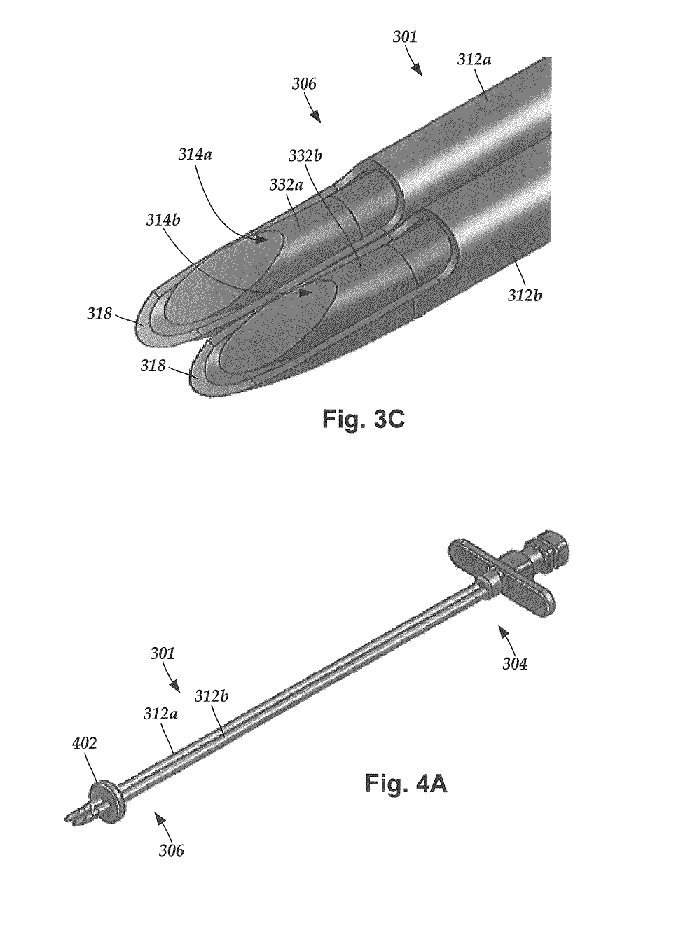

Turning to FIGS. 3A-5B, in at least some embodiments the multi-lead introducer includes multiple individual needles joined to a common hub. FIG. 3A is a schematic perspective view of one embodiment of a multi-lead introducer 300 configured and arranged for concurrently inserting multiple leads (e.g., leads 103a and 103b of FIG. 1) into a patient FIG. 3B is a schematic perspective exploded view of one embodiment of the multi-lead introducer 300. FIG. 3C is a schematic close-up, perspective view of one embodiment of a distal end of the multi-lead introducer 300.

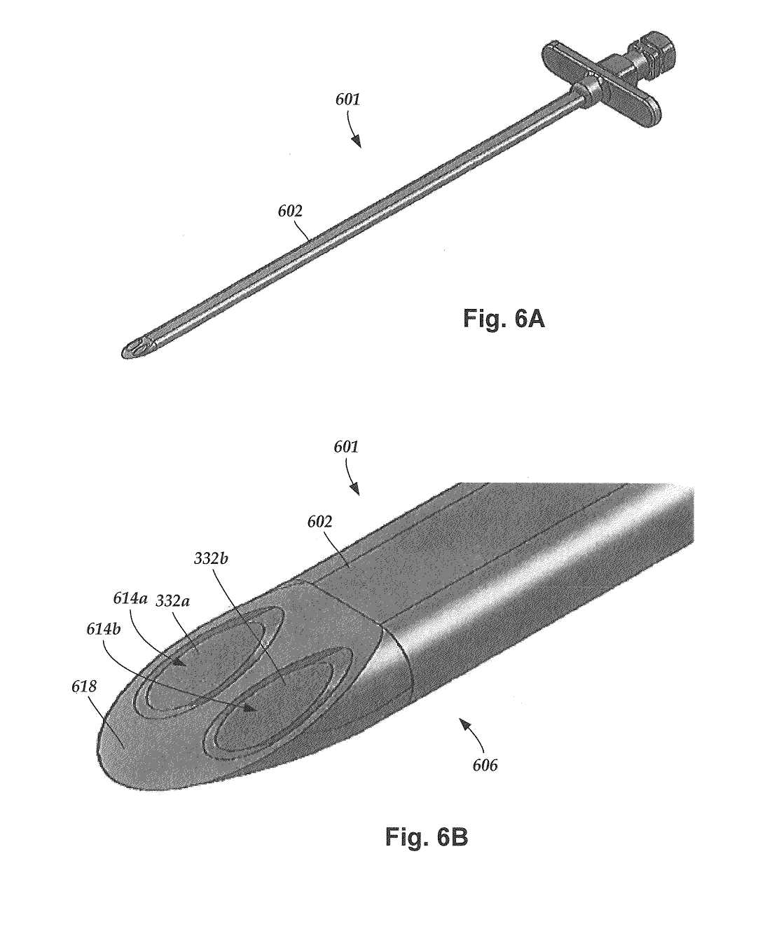

The multi-lead introducer 300 includes a needle assembly 301 having a proximal end 304 and a distal end 306. A hub 320 is coupled to the proximal end 304 of the needle assembly 301. In FIGS. 3A-3C, the needle assembly 301 is shown as including two individual needles 312a and 312b. It will be understood that the needle assembly 301 may include any suitable number of needles including, for example, one, two, three, four, five, six, or more needles. FIGS. 6A-9, for example, illustrate embodiments of the needle assembly that include a single needle.

In at least some embodiments, the needles 312a and 312b are positioned in a side-by-side arrangement. In at least some embodiments, the needles 312a and 312b are in physical contact with one another along at least a portion of the longitudinal length of the needle assembly 301, while in at least some other embodiments the needles 312a. and 312b are positioned with a gap between the needles 312a and 312b such that the needles 312a and 312b do not physically touch one another along the entire longitudinal length of the needle assembly 301. It will be understood that, although the needles may or may not physically contact one another along the longitudinal length of the needle assembly 301, the needles 312a and 312b are each physically coupled to the hub 320 at the proximal end 304 of the needle assembly.

The needles 312a and 312b can be formed from any material suitable for implantation including, for example, one or more metals (e.g., stainless steel, Nitinol™, titanium, or the like), one or more alloys, one or more plastic resins, or the like. Each of the needles of the needle assembly 301 defines one or more needle lumens extending from the proximal end 304 to the distal end 306. In FIGS. 3A-3C, each of the two individual needles 312a and 312b defines an individual needle lumen 314a and 314b, respectively. It will be understood that each individual needle of the needle assembly 301 may include any suitable number of needle lumens including, for example, one, two, three, four, or more needle lumens.

The needles 312a and 312b have any selected bore size suitable for receiving a lead (see e.g., 103 in FIG. 1) including, for example, 14-gauge, 15-gauge, 16-gauge, 17-gauge, 18-gauge, 19-gauge, or larger. In at least some embodiments, the distal end 306 of the needle assembly 301 includes one or more distal tips 318 (FIG. 3C) that form one or more sharpened surfaces that facilitate initiating a path through patient tissue during insertion of the multi-lead introducer 300 into a patient. In FIG. 3C, each of the needles 312s and 312b includes a separate distal tip 318. In at least some embodiments, the one or more distal tips 318 are beveled. In at least some embodiments, insertion three for the needles 312a, 312b is reduced/controlled by applying, a silicone lubricant to the one or more distal tips 318 or by making the one or more distal tips 318 out of a lubricious material, such as fluoropolymers, or coating the metal distal tip 318 in a lubricious coating or plating.

Any suitable huh 320 may be coupled to the needle assembly 301. In FIGS. 3A-3B (and in other figures), the hub 320 is shown as a proximal female Luer hub that is coupled to the proximal ends 304 of each of the one or more needles 312a and 312b. In at least some embodiments, the hub 320 includes one or more handles 322.

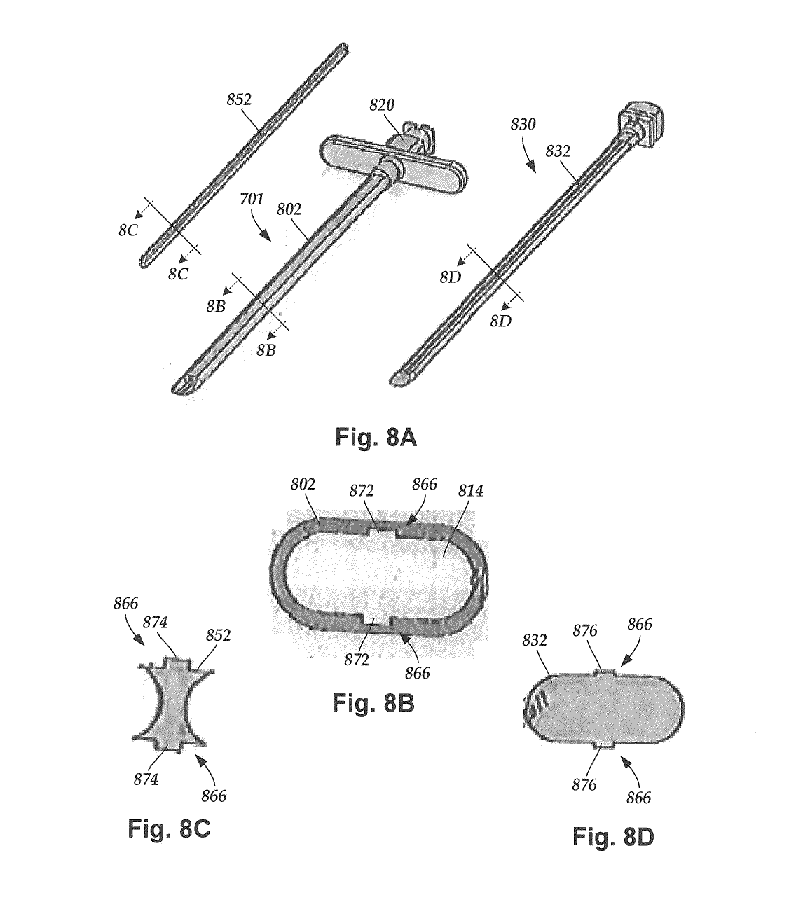

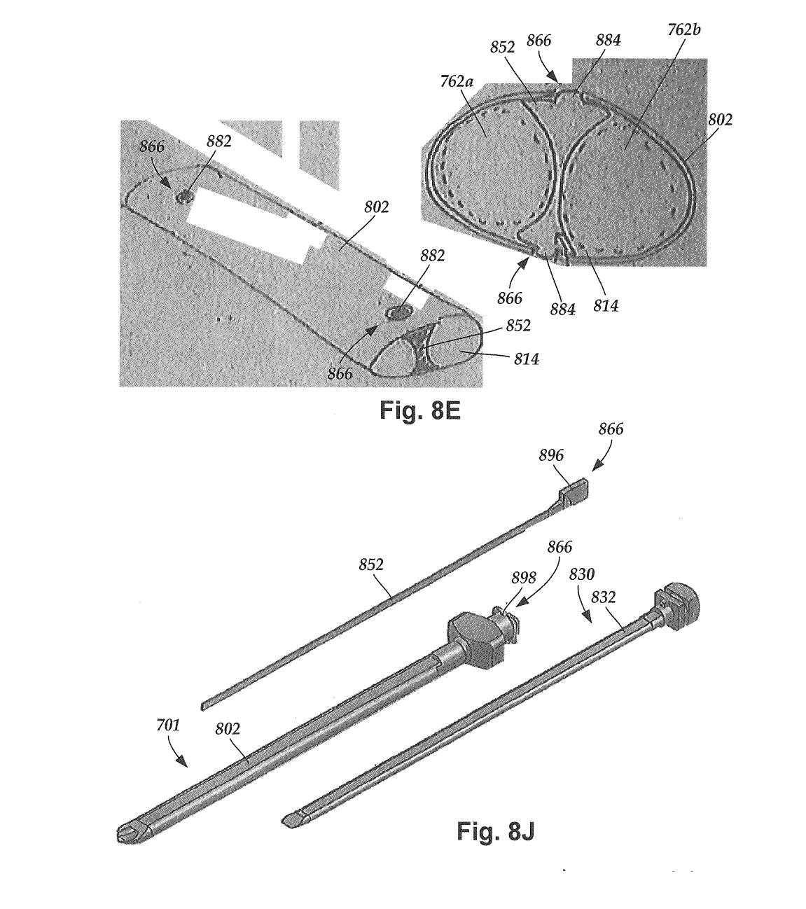

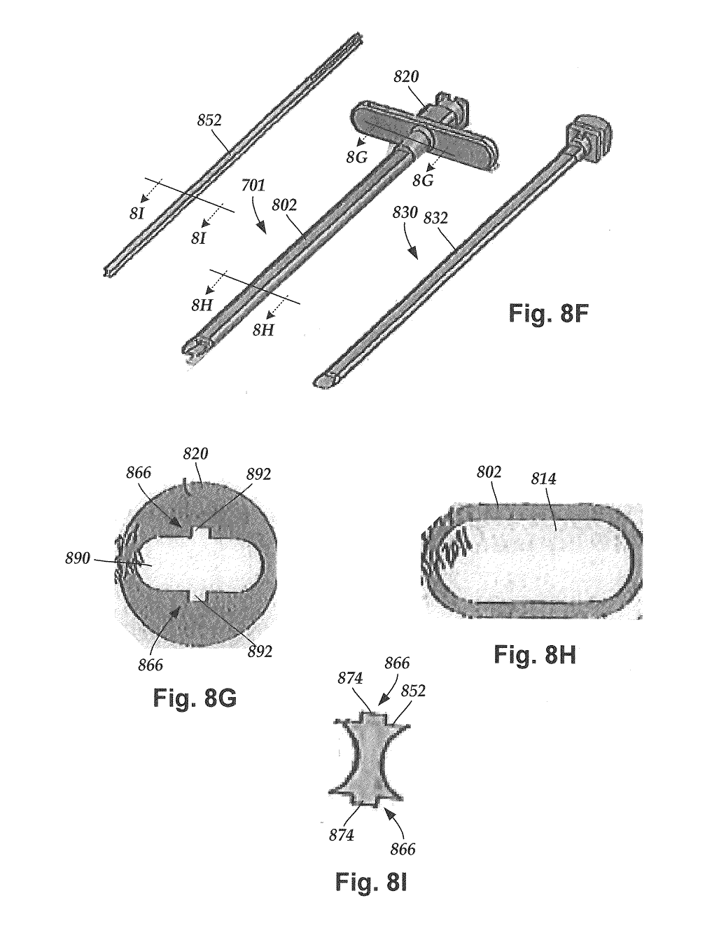

The hub 320 is configured and arranged to simultaneously receive multiple leads so that each different lead of the multiple leads can be simultaneously extended through the hub 320 and inserted into the one or more needle lumens. In at least some embodiments, the hub 320 defines a single hub lumen configured to concurrently receive multiple leads (see e.g., 890 in FIG. 8G). In at least some alternate embodiments, the hub 320 defines multiple hub lumens, where each of the multiple hub lumens is configured to receive one or more leads. The one or more hub lumens are in communication with the one or more needle lumens such that when a lead is extended through one of the one or more hub lumens, the lead can be further extended through one of the one or more needle lumens.

The hub 320 can be formed from any material suitable for implantation including, for example, one or more metals (e.g., stainless steel, Nitinol™, titanium, or the like), one or more alloys, one or more plastic resins, or the like. Other molding or formation techniques can also be used. The hub 320 can be coupled to the one or more needles in any suitable manner including, for example, welding, bonding, brazing, insert molding (e.g., using an insert molded thermoplastic), or the like or combinations thereof.

In at least some embodiments, the hub 320 includes a mating male Luer tip syringe configured and arranged for injecting or withdrawing fluid or air during insertion of the multi-lead introducer. For example, during insertion of the multi-lead introducer, fluid (e.g., saline solution, air, or the like) may be introduced or removed through the luer hub 320 to check for positioning of the multi-lead introducer 300 (e.g., in an epidural space of the patient).

Optionally, the multi-lead introducer includes one or more stylets 330 (FIG. 313) configured and arranged for insertion into the needle assembly 301 during a lead implantation procedure. In at least some embodiments, the one or more stylets 330 are configured and arranged for insertion into the one or more needle lumens and the one or more hub lumens. In at least some embodiments, the one or more stylets 330 include one or more stylet rods each having a proximal end and a distal end, where the proximal end is coupled to a common stylet handle (or separate stylet handle), or stylet hub, and the distal end is preferably configured and arranged to reduce the risk of tissue coring during insertion of the needle assembly 301 when the one or more stylet rods are inserted into the one or more needle lumens.

In FIGS. 3A-3C, the stylet 330 is shown including a first stylet rod 332a for insertion into the needle lumen 314a and a second stylet rod 332b for insertion into the needle lumen 314b. In FIGS. 3A-3C, the stylet 330 also includes a single stylet handle 334 coupled to each of the stylet rods 332a and 332b. In at least some alternate embodiments, multiple stylets may be used in lieu of the single stylet 330. In which case, when the needle assembly 301 defines multiple needle lumens, each individual stylet can be inserted into a different one of the multiple needle lumens.

The stylet 330 can be formed from any material suitable for implantation including, for example, one or more metals (e.g., stainless steel, Nitinol™, titanium, or the like), one or more alloys, one or more plastic resins, or the like.

The stylet handle (or handles) 334 can be formed from any material suitable for implantation including, for example, one or more metals (e.g., stainless steel, Nitinol™, titanium, or the like), one or more alloys, one or more plastic resins, or the like. In at least some embodiments, the stylet handle 334 is insert molded onto a metal stylet(s) using an insert molded thermoplastic.

The multi-lead introducer can be inserted into the patient in proximity to a target stimulation location (e.g., the epidural space of the patient). Optionally, the stylet(s) are inserted into the needle assembly to reduce the risk of tissue coring. The needle assembly and stylet(s) are inserted into the patient and guided into proximity of the target stimulation location.

Once the needle assembly and stylet(s) are in proximity to a target stimulation location, the positioning of the needle assembly may be checked. The positioning of the needle assembly may be checked in any suitable manner, such as by introducing or removing fluid through the luer hub 320 (e.g., performing a loss of resistance test), imaging (e.g., via fluoroscopy, magnetic resonance imaging, or the like) the patient with or without introducing one or more contrast agents into the patient, or using the electrodes of the lead (or another insertable stimulation device) to stimulate surrounding patient tissue.

Once the needle assembly and stylet(s) are in proximity to the target stimulation location, the stylet(s) 330 are removed and the leads are advanced along the needle assembly. The leads can be advanced along the needle assembly either sequentially or concurrently. It may be advantageous for the leads to be physically separated from one another when the leads are advanced out from the distal end of the needle assembly and into the patient so that the leads can be moved independently of one another when positioning the leads within the patient.

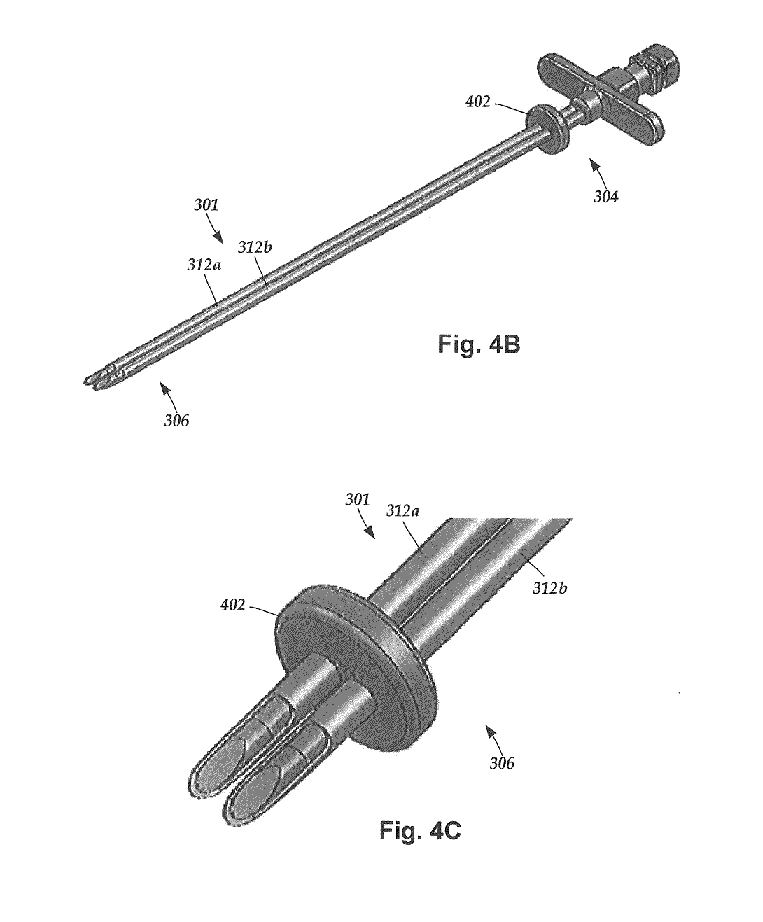

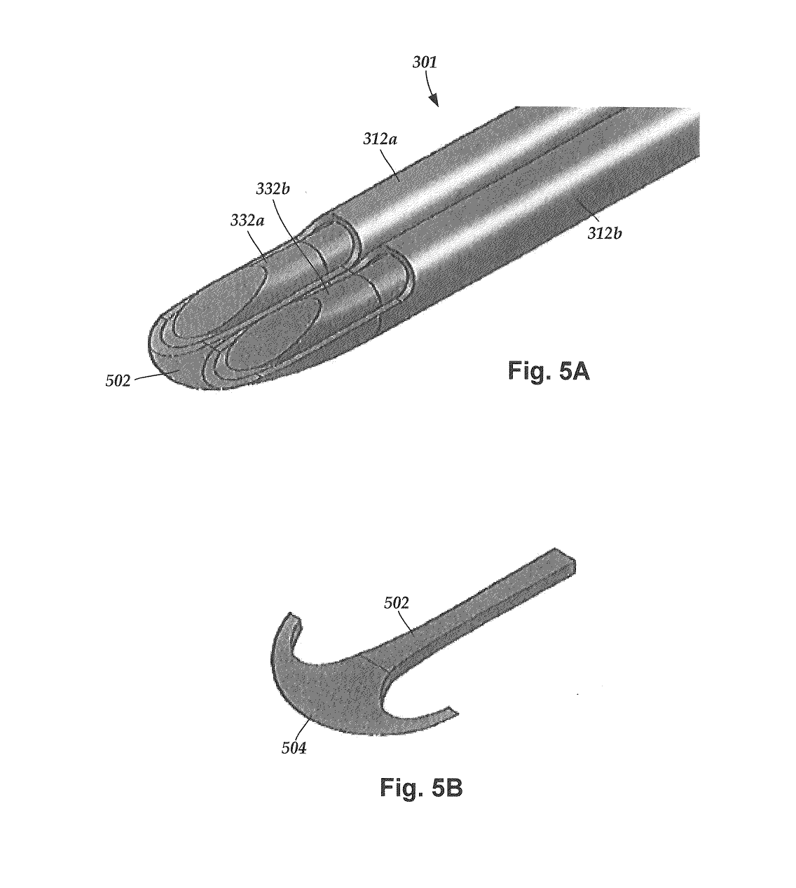

Turning to FIGS. 4A-5B, in at least some multiple-needle embodiments the needles are physically coupled to one another along one or more locations along the longitudinal length of the needle assembly. In at least some embodiments, the needles are welded or bonded together at one or more locations. In at least some embodiments, the multiple needles are physically coupled together at the distal end of the needle assembly.

In at least some embodiments the multi-lead introducer includes one or more needle couplers disposed over the needles to modulate physical coupling between the individual needles, along the longitudinal lengths of the needles. In at least some embodiments, the needle coupler 402 is configured and arranged to prevent, or hinder, the distal tips of the needles from spreading away from each other during handling/insertion.

FIG. 4A is a schematic perspective view of one embodiment of a needle coupler 402 disposed over a portion of the distal end 306 of the needle assembly 301.

FIG. 4B is a schematic perspective view of one embodiment of the needle coupler 402 disposed over a portion of the proximal end 304 of the needle assembly 301. FIG. 4C is a schematic close-up, perspective view of one embodiment of the needle coupler 402 disposed over a portion of the distal end 306 of the needle assembly 301.

In at least some embodiments, the needle coupler 402 is disposed over a portion of the distal end 306 of the needle assembly 301 such that the needle coupler 402 is coupled to each of the needles of the needle assembly. In at least some alternate embodiments, the needle coupler 402 is coupled to less than all of the needles of the needle assembly. The needle coupler 402 can be coupled to the needle assembly in any suitable manner. In at least some embodiments, the needle coupler 402 is press-fit over the needle assembly.

In at least some embodiments, the needle couple 402 is configured and arranged to slidably move along the longitudinal length of the needle assembly 301. In at least some embodiments, the needle coupler 402 is initially positioned at the distal end 306 of the needle assembly 301 to maintain the needles 312a, 312b in close proximity to one another during insertion, and moves proximally during insertion by the pressure exerted on the needle coupler 402 by the patient's skin.

The needle coupler 402 can be formed from any suitable material including, for example, an elastomer (e.g., rubber, silicone, plastic resin, stamped metal, a wire spring element, or the like or combinations thereof). In at least some embodiments, the needle coupler 402 maintains attachment to the needle assembly 301, while also being slidably moveable along the longitudinal length of the needle assembly 301 during insertion into the patient.

Turning to FIGS. 5A-5B, in at least some embodiments the multiple needles are physically (optionally, permanently) coupled to one another along the distal tips of the needles. FIG. 5A is a schematic perspective, close-up view of one embodiment of a distal tip insert 502 coupled to the distal tips 318 of each of the needles 312a, 312b of the needle assembly 301. FIG. 5B is a schematic perspective view of one embodiment of the distal tip insert 502. The distal tip insert 502 can include a single sharpened region 504 that can function to transform multiple penetrating surfaces of the needles into a single penetrating surface for contacting patient tissue during implantation.

The distal tip insert 502 can be formed from any hard material that is suitable for tissue cutting/dissection. In at least some embodiments, the distal tip insert 502 is formed from a metal that is capable of being joined (e.g., welded) to the needles. Such a configuration may improve manufacture by simplifying manufacture, or reducing the cost of manufacture, or both.

The distal tip insert 502 can be either temporary or permanent. The distal tip insert 502 can be coupled to the distal tips of the individual needles in any suitable manner including, for example, friction fit, adhesive, welding, brazing, or the like or combinations thereof.

Turning to FIGS. 6A-6B, in at least some embodiments the multi-lead introducer includes a needle assembly with a single needle that defines multiple discrete needle lumens. Different needle assemblies can be designed for any suitable number of leads. In at least some embodiments, each individual needle lumen is configured and arranged to receive a single lead.

FIG. 6A is a schematic perspective view of one embodiment of a needle assembly 601 formed from a single needle 602. FIG. 6B is a schematic perspective, close-up view of one embodiment of a distal end 606 of the single-needle needle assembly 601. The single needle 602 defines multiple needle lumens 614a and 614b. In FIGS. 6A-6B, the distal ends of the stylet rods 332a and 332b are shown disposed in the needle lumens 614a and 614h, respectively. In at least some embodiments, a distal tip 618 incorporates a distal bevel with a single point to reduce the force to introduce the needle 602 into the patient.

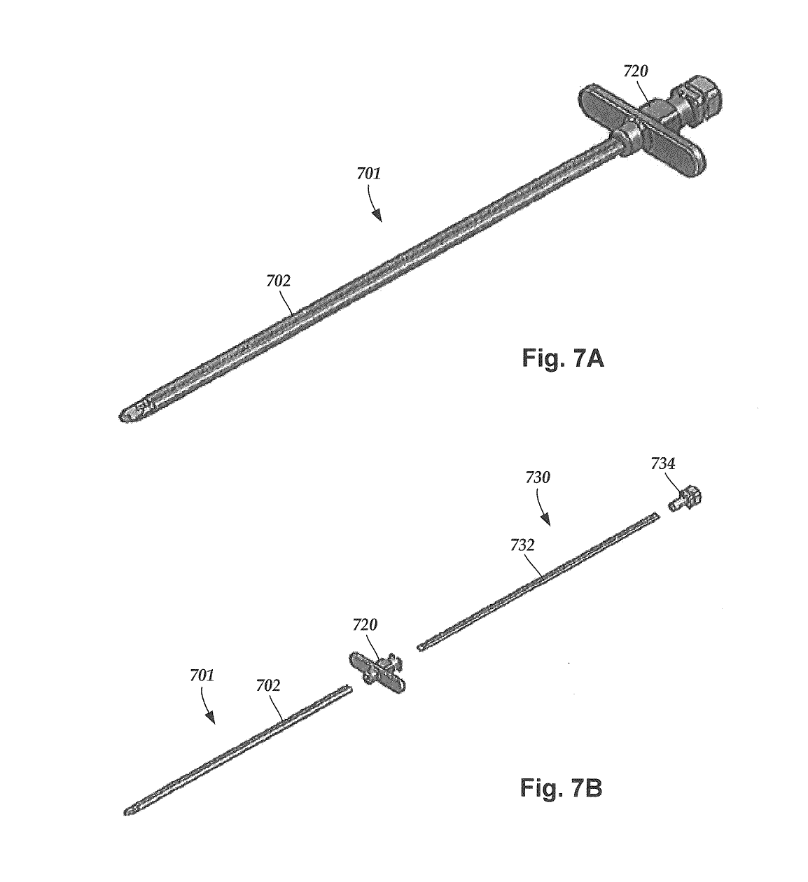

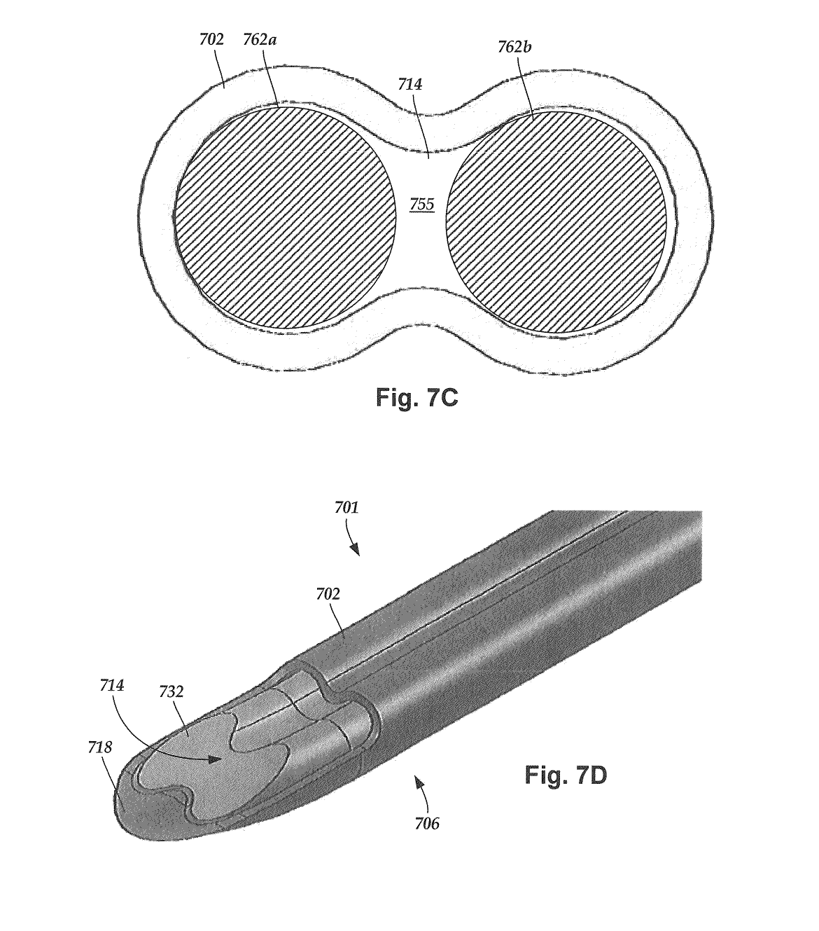

Turning to FIGS. 7A-8D, in at least some embodiments the needle assembly includes a single needle that defines a single lumen, where the single lumen is configured and arranged to receive multiple leads. FIG. 7A is a schematic perspective view of one embodiment of a needle assembly 701 that includes a single needle 702 coupled to a hub 720. FIG. 7B is a schematic perspective, exploded view of one embodiment of the needle assembly 701 and a stylet 730 configured and arranged for insertion into the needle 702 of the needle assembly 701. The stylet 730 includes a stylet rod 732 coupled on one end to a stylet handle 734.

FIG. 7C is a schematic transverse cross-sectional view of one embodiment of the needle 702, where the needle 702 defines a needle lumen 714 configured and arranged to receive multiple leads 762a and 762b. In at least some embodiments, the needle lumen 714 is configured and arranged to receive the leads 762a and 762b such that the leads 762a and 762b do not physically touch one another within the needle lumen 714. In FIG. 7C, the needle lumen 714 is shown defining a lead separation region 755 defined between the leads 762a and 762b when the leads 762a and 762b are disposed in the needle lumen 714. In at least some embodiments, the lead separation region 755 is bounded on at least one side by a narrowing of the needle lumen 714 which functions to separate the leads 762a and 762b and prevent or reduce the leads from physically contacting one another when disposed in the needle lumen 714. In FIG. 7C, the narrowing of the needle lumen 714 is shown being formed by two opposing convex when viewed from inside the needle) regions of a transverse perimeter of the needle lumen 714. As mentioned above, it may be advantageous for the leads to not physically touch one another within the needle assembly during an implantation procedure so that the leads can be moved independently of one another.

Additionally, the needle lumen 714 is also configured and arranged to receive the stylet rod 732. For example, during an implantation procedure the needle 702 may be inserted into the patient while the stylet rod 732 is disposed in the needle lumen 714. FIG. 7D is a schematic perspective, close-up view of one embodiment of a distal portion of the stylet rod 732 disposed in a distal end 706 of the needle 702. In at least some embodiments, a distal tip 718 incorporates a distal bevel with a single point to reduce the force needed to introduce the needle 702 into the patient.

In at least some embodiments, the transverse shape of the stylet rod 732 is similar to the transverse shape of the needle lumen 714. In FIG. 7D, the stylet rod 732 is shown having a transverse shape that includes two opposing concave regions that align with the two opposing convex regions of the needle lumen 714.

Turning to FIGS. 8A-8J, in at least some embodiments one or more dividers are disposed along at least at least a portion of the longitudinal length of the needle assembly. The one or more dividers function to maintain a separation between multiple leads when multiple leads are disposed in a particular lumen of the needle assembly. In at least some embodiments, the one or more dividers may be removable so that the one or more dividers are inserted into the needle lumen when the one or more leads are disposed in the needle lumen, but are removed when, for example, the stylet rod is inserted into the needle lumen.

FIG. 8A is a schematic perspective view of another embodiment of the needle assembly 701 that includes a single needle 802 coupled to a hub 820, a stylet 830 with a stylet rod 832 configured for insertion into the needle assembly 701, and a divider 852 also configured for insertion into the needle assembly 701.

As discussed above, in at least some embodiments the divider 852 is configured and arranged to be removable so that the divider 852 is disposed in the needle assembly 701 when the leads are disposed in the needle assembly 701 to physically separate the leads from one another, but not present in the needle assembly 701 when the stylet rod 832 is disposed in the needle assembly 701.

Turning to FIGS. 8B-8D, in at least some embodiments a retention assembly 866 is used to maintain the positioning of the divider 852 (or the stylet rod 832) when the divider 852 (or the stylet rod 832) is disposed in the needle assembly 701. In at least some embodiments, the retention assembly 866 includes a first element disposed on a first component and a second element that mates, or interlocks, with the first element and that is disposed on a second component. For example, in FIGS. 8B-8D the retention assembly 866 includes a first element disposed along at least a portion of the needle assembly 701 and a second element that mates with the first element and that is disposed along the divider 852 (or the stylet 832).

FIGS. 8B-8D illustrate transverse cross-sectional views of embodiments of the components of the multi-lead introducer shown in FIG. 8A. In FIGS. 8B-8D, the retention assembly 866 includes slots and tabs configured and arranged to mate with the slots. FIG. 8B illustrates a transverse cross-sectional view of one embodiment of the needle 802. A needle lumen 814 is defined along a length of the needle 802. In FIG. 813, the needle lumen 814 is shown defining slots 872 along opposing edges of the needle lumen 814.

FIG. 8C illustrates a transverse cross-sectional view of one embodiment of the divider 852. The divider 852 includes tabs 874 disposed on opposing ends of the divider 852. The tabs 874 are configured and arranged to mate with the slots 872 (of the needle lumen 814) when the divider 852 is disposed in the needle lumen 814.

FIG. 8D illustrates a transverse cross-sectional view of one embodiment of the stylet rod 832. The stylet rod 832 includes tabs 876 disposed on opposing ends of the stylet rod 832. The tabs 876 are configured and arranged to mate with the slots 872 (of the needle lumen 814) when the stylet rod 832 is disposed in the needle lumen 814.

It will be understood that, in alternate embodiments of the retention assembly 866 the tabs are disposed in needle lumen 814 and the slots are defined along the divider 852, or the stylet rod 832, or both. In at least some other embodiments, the needle lumen 814 and at least one of the divider 852 or the stylet rod 832 includes both at least one slot and at least one tab.

In at least some embodiments, the retention assembly includes apertures and protrusions configured to mate with the apertures. FIG. 8E is schematic perspective view of one embodiment of the divider 852 disposed in the needle lumen 814 of the needle 802. FIG. 8E also illustrates a transverse cross-sectional view of one embodiment of the two leads 762a and 762b disposed in the needle lumen 814 of needle 802, with the two leads 762a and 762b separated from one another by the divider 852.

In FIG. 8E, the needle lumen 814 is shown defining apertures 882 along opposing edges of the needle lumen 814. FIG. 8E also shows the apertures 882 defined in proximity to opposing ends of the needle lumen 814. The divider 852 includes protrusions 884 disposed along the divider 852. The protrusions 884 are configured and arranged to mate with the apertures 882 when the divider 852 is disposed in the needle lumen 814. In FIG. 8E, the apertures 882 are shown extending through to an outer surface of the needle 802. In alternate embodiments, the apertures 882 do not extend through to the outer surface of the needle 802.

In at least some embodiments, the element of the retention assembly 866 disposed along the needle assembly 701 is disposed, either partially or entirely, along the hub of the needle assembly. FIG. 8F is a schematic perspective view of yet another embodiment of the needle assembly 701 that includes the single needle 802 coupled to the hub 820, the stylet 830 with the stylet rod 832 configured for insertion into the needle assembly 701, and the divider 852 also configured for insertion into the needle assembly 701.

FIGS. 8G-8I illustrate transverse cross-sectional views of embodiments of the components of the multi-lead introducer shown in FIG. 8E. In FIGS. 8G-8I, the retention assembly 866 includes slots and tabs configured and arranged to mate with the slots. FIG. 80 illustrates a transverse cross-sectional view of one embodiment of the hub 820. A hub lumen 890 is defined along the hub 820. The hub lumen 890 is shown defining slots 892 along opposing edges of the hub lumen 890.

FIG. 8H illustrates a transverse cross-sectional view of one embodiment of the needle 802. A needle lumen 814 is defined along the needle 802. The needle lumen 814 and the hub lumen 890 are in communication with one another such that, when the divider 852 (or stylet rod 832) is extended along the needle assembly 701, the divider 852 or stylet rod 832 extends along both the needle lumen 814 and the hub lumen 890. In FIG. 8H, the needle lumen 814 is shown without slots. In at least some embodiments, the needle lumen 814 defines slots.

FIG. 8I illustrates a transverse cross-sectional view of one embodiment of the divider 852. The divider 852 includes tabs 874 disposed on opposing ends of the divider 852. The tabs 874 are configured and arranged to mate with the slots 892 (of the hub lumen 890) when the divider 852 is disposed in the hub lumen 890. In embodiments where the needle lumen 814 also defines slots, the tabs 874 may, additionally, be configured and arranged to mate with those slots (of the needle lumen 814) when the divider 852 is disposed in the needle lumen 814.

In embodiments where the needle lumen 814 does not include slots (as is shown in FIG. 8H), when the divider 852 is disposed in the needle assembly 701 the divider 852 maintains positioning within both the needle lumen 814 and the hub lumen 890 using slots 892 defined in the hub lumen 890.

It will be understood that, in alternate embodiments, the tabs are disposed in the hub lumen 890 and the slots are defined along the divider 852. In at least some other embodiments, each of the hub lumen 890 and the divider 852 includes both at least one slot and at least one tab.

In at least some embodiments, the element of the retention assembly 866 disposed along the needle assembly 701 is disposed, either partially or entirely, external to the needle assembly. FIG. 8J is a schematic perspective view of another embodiment of the needle assembly 701 that includes the single needle 802 coupled to the hub 820, the stylet 830 with the stylet rod 832 configured for insertion into the needle assembly 701, and the divider 852 also configured for insertion into the needle assembly 701.

In FIG. 8J, the divider 852 includes a tab 896 disposed at one end of the divider 852, and the hub 820 defines a notch 898. The tab 896 is configured and arranged to mate with the notch 898 when the divider 852 is disposed in the needle assembly 701. In at least some embodiments, the tab 896 does not mate with the notch 898 until the divider 852 is fully inserted into the needle assembly 701.

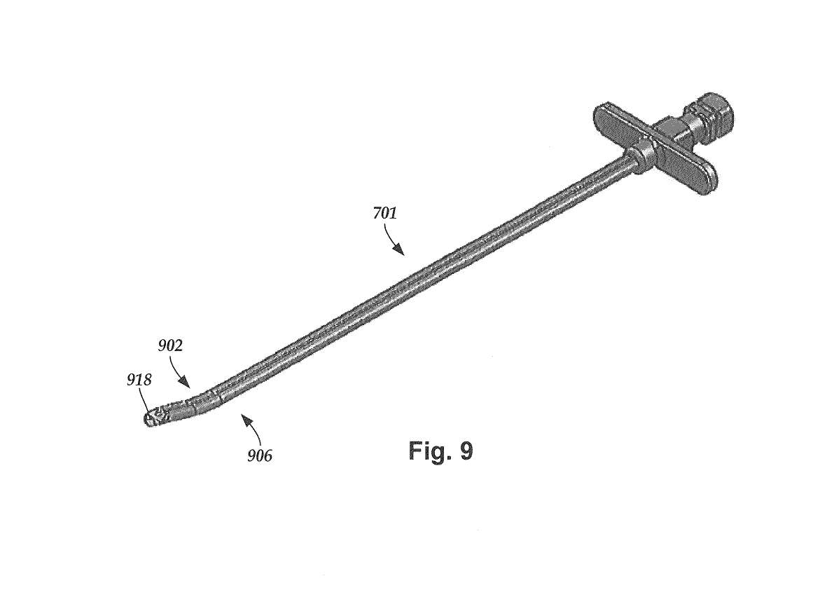

Optionally, in at least some embodiments the needle assembly includes one or more bends. FIG. 9 is a schematic perspective view of one embodiment of a bend 902 formed along the needle assembly 701. In at least some embodiments, the bend 902 is formed along the distal end 306 of the needle assembly 701. In at least some embodiments, the bend 902 is formed along a distal end 906 of the needle assembly 701 proximal to a distal tip 918.

The bend 902 is configured and arranged to alter the approach angle of the needle assembly 701 during lead implantation. In at least some embodiments, altering the approach angle of the needle assembly 701 facilitates lead implantation. The bend 902 can be any suitable angle. In at least some embodiments, the bend 902 is at least 5°. In at least some embodiments, the bend 902 is no greater than 20°. In at least some embodiments, the bend 902 is at least 5° and no greater than 15°, it will be understood that the bend 902 can be formed along any of the disclosed embodiments of the needle assembly. It will also be understood that the needle assembly may include additional bends, as desired.

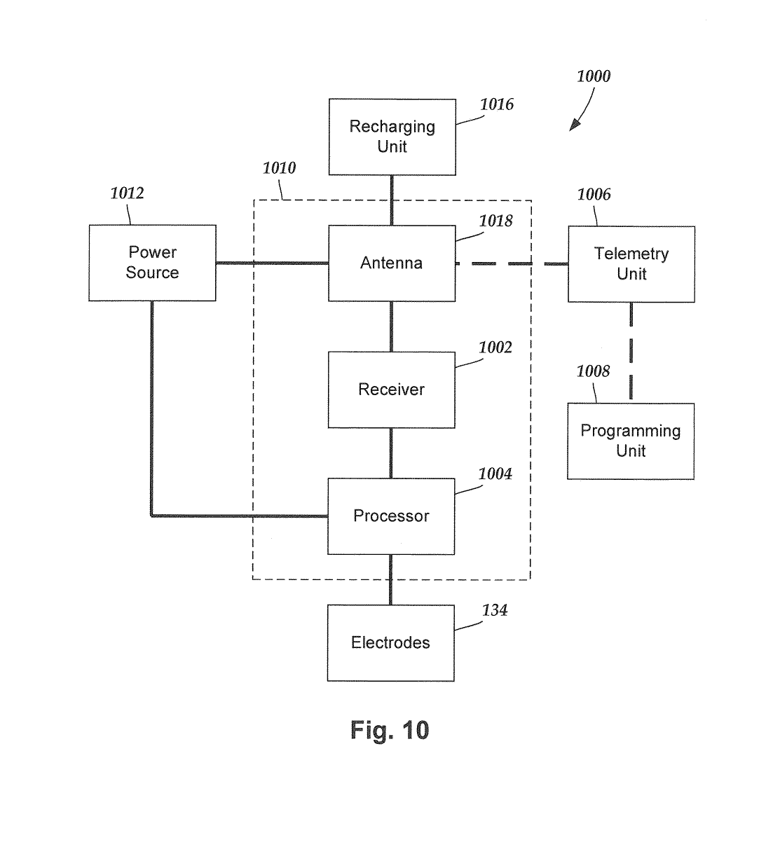

FIG. 10 is a schematic overview of one embodiment of components of an electrical stimulation system 1000 including an electronic subassembly 1010 disposed within a control module. It will be understood that the electrical stimulation system can include more, fewer, or different components and can have a variety of different configurations including those configurations disclosed in the stimulator references cited herein.

Some of the components (for example, power source 1012, antenna 1018, receiver 1002, and processor 1004) of the electrical stimulation system can be positioned on one or more circuit boards or similar carriers within a sealed housing of an implantable pulse generator, if desired. Any power source 1012 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1018 or a secondary antenna. The external power source can be in a device that is mounted on the skin of the user or in a unit that is provided near the user on a permanent or periodic basis.

If the power source 1012 is a rechargeable battery, the battery may be recharged using the optional antenna 1018, if desired. Power can be provided to the battery for recharging by inductively coupling the battery through the antenna to a recharging unit 1016 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1004 is generally included to control the timing and electrical characteristics of the electrical stimulation system. For example, the processor 1004 can, if desired, control one or more of the timing, frequency, strength, duration, and waveform of the pulses. In addition, the processor 1004 can select which electrodes can be used to provide stimulation, if desired. In some embodiments, the processor 1004 may select which electrode(s) are cathodes and which electrode(s) are anodes. In some embodiments, the processor 1004 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1508 that, for example, allows modification of pulse characteristics. In the illustrated embodiment, the processor 1004 is coupled to a receiver 1002 which, in turn, is coupled to the optional antenna 1018. This allows the processor 1004 to receive instructions from an external source to, for example, direct the pulse characteristics and the selection of electrodes, if desired.

In one embodiment, the antenna 1018 is capable of receiving signals (e.g., RF signals) from an external telemetry unit 1006 which is programmed by a programming unit 1008. The programming unit 1008 can be external to, or part of, the telemetry unit 1006. The telemetry unit 1006 can be a device that is worn on the skin of the user or can be carried by the user and can have a form similar to a pager, cellular phone, or remote control, if desired. As another alternative, the telemetry unit 1006 may not be worn or carried by the user but may only be available at a home station or at a clinician's office. The programming unit 1008 can be any unit that can provide information to the telemetry unit 1006 for transmission to the electrical stimulation system 1000. The programming unit 1008 can be part of the telemetry unit 1006 or can provide signals or information to the telemetry unit 1006 via a wireless or wired connection. One example of a suitable programming unit is a computer operated by the user or clinician to send signals to the telemetry unit 1006.

The signals sent to the processor 1004 via the antenna 1018 and receiver 1002 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1000 to cease operation, to start operation, to start charging the battery, or to stop charging the battery. In other embodiments, the stimulation system does not include an antenna 1018 or receiver 1002 and the processor 1004 operates as programmed.

Optionally, the electrical stimulation system 1000 may include a transmitter (not shown) coupled to the processor 1004 and the antenna 1018 for transmitting signals back to the telemetry unit 1006 or another unit capable of receiving the signals. For example, the electrical stimulation system 1000 may transmit signals indicating whether the electrical stimulation system 1000 is operating properly or not or indicating when the battery needs to be charged or the level of charge remaining in the battery. The processor 1004 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A multi-lead introducer for facilitating implantation of at least one electrical stimulation lead, the multi-lead introducer comprising:
a needle assembly configured and arranged to concurrently implant a plurality of leads into a patient, the needle assembly comprising
a first needle having a first end, an opposing second end, and a longitudinal length, the first needle defining a first needle lumen extending along the longitudinal length of the first needle from the first end to the second end, the first needle lumen configured and arranged to receive a first lead of the plurality of leads,
a first sharpened tip disposed along the first end of the first needle, the first sharpened tip configured and arranged to pierce patient tissue,
a second needle having a first end, an opposing second end, and a longitudinal length, the second needle defining a second needle lumen extending along the longitudinal length of the second needle from the first end to the second end, the second needle lumen configured and arranged to receive a second lead of the plurality of leads,
a second sharpened tip disposed along the first end of the second needle, the second sharpened tip configured and arranged to pierce patient tissue, and
a hub coupled to both the second end of the first needle and the second end of the second needle, the hub defining at least one hub lumen that is in communication with both the first needle lumen and the second needle lumen, the at least one hub lumen configured and arranged to concurrently receive both the first lead and the second lead.

2. The multi-lead introducer of claim 1, wherein the first needle and the second needle are disposed in a side-by-side arrangement.

3. The multi-lead introducer of claim 2, wherein the first needle and the second needle are disposed in a side-by-side arrangement with the first needle physically spaced-apart from the second needle.

4. The multi-lead introducer of claim 2, wherein the first needle and the second needle are disposed in a side-by-side arrangement with at least a portion of the longitudinal length of the first needle physically coupled to at least a portion of the longitudinal length of the second needle.

5. The multi-lead introducer of claim 4, wherein at least a portion of the longitudinal length of the first needle is welded or bonded to at least a portion of the longitudinal length of the second needle.

6. The multi-lead introducer of claim 4, further comprising at least one needle coupler disposed over each of the first needle and the second needle, the at least one needle coupler configured and arranged to move along the longitudinal lengths of the first needle and the second needle to modulate the location of the physical coupling between the first needle and the second needle along the longitudinal lengths of the first needle and the second needle.

7. The multi-lead introducer of claim 1, further comprising a distal tip insert with a sharpened distal region, the distal tip insert configured and arranged to couple to both the first needle and the second needle and to form a common distal tip with the first distal tip and the second distal tip.

8. The multi-lead introducer of claim 1, further comprising a stylet configured and arranged for concurrent insertion into both the first needle lumen and the second needle lumen, the stylet configured and arranged for reducing tissue coring during insertion of the needle assembly into the patient.

9. The multi-lead introducer of claim 8, wherein the stylet comprises a first stylet rod configured and arranged for insertion into the first needle lumen, a second stylet rod configured and arranged for insertion into the second needle lumen, and a stylet handle coupled to both the first stylet rod and the second stylet rod.

10. An insertion kit for facilitating implantation of an electrical stimulation lead, the insertion kit comprising:
the multi-lead introducer of claim 1;
a first lead comprising
a first lead body having a distal end, a proximal end, and a longitudinal length,
a plurality of first electrodes disposed along the distal end of the first lead body,
a plurality of first terminals disposed along the proximal end of the first lead body, and
a plurality of first conductors, each first conductor electrically coupling at least one of the first electrodes to at least one of the first terminals;
a second lead comprising
a second lead body having a distal end, a proximal end, and a longitudinal length,
a plurality of second electrodes disposed along the distal end of the second lead body,
a plurality of second terminals disposed along the proximal end of the second lead body, and
a plurality of second conductors, each second conductor electrically coupling at least one of the second electrodes to at least one of the second terminals;
wherein the first lead is configured and arranged for insertion into the first needle lumen of the multi-lead introducer; and
wherein the second lead is configured and arranged for insertion into the second needle lumen of the multi-lead introducer.

11. An electrical stimulating system comprising:
the insertion kit of claim 10;
a control module configured and arranged to electrically couple to both the proximal end of the first lead body of the insertion kit and the proximal end of the second lead body of the insertion kit, the control module comprising
a housing, and
an electronic subassembly disposed in the housing; and
a connector for receiving both the first lead of the insertion kit and the second lead of the insertion kit, the connector having a proximal end, a distal end, and a longitudinal length, the connector comprising
a connector housing defining a first port and a second port at the distal end of the connector, the first port configured and arranged for receiving the proximal end of the first lead body of the insertion kit, the second port configured and arranged for receiving the proximal end of the second lead body of the insertion kit,
a first plurality of connector contacts disposed in the connector housing, the first plurality of connector contacts configured and arranged to couple to at least one of the plurality of first terminals disposed on the proximal end of the first lead body of the insertion kit, and
a second plurality of connector contacts disposed in the connector housing, the second plurality of connector contacts configured and arranged to couple to at least one of the plurality of second terminals disposed on the proximal end of the second lead body of the insertion kit.

12. A multi-lead introducer for facilitating implantation of at least one electrical stimulation lead, the multi-lead introducer comprising:
a needle assembly configured and arranged to concurrently implant a plurality of leads into a patient, the needle assembly comprising
a single needle having a first end, an opposing second end, and a longitudinal length, the single needle defining a single needle lumen extending along the longitudinal length of the single needle from the first end to the opposing second end, the single needle lumen configured and arranged to concurrently receive both a first lead and a second lead of the plurality of leads,
a sharpened tip disposed along the first end of the single needle, the sharpened tip configured and arranged to pierce patient tissue, and
a hub coupled to the second end of the single needle, the hub defining at least one hub lumen that is in communication with the single needle lumen, the at least one hub lumen configured and arranged to concurrently receive both the first lead and the second lead.

13. The multi-lead introducer of claim 12, wherein the single needle lumen is configured and arranged to concurrently receive the first lead of the plurality of leads and the second lead of the plurality of leads with the first lead and the second lead being physically spaced apart from one another.

14. The multi-lead introducer of claim 12, further comprising at least one divider extending along at least a portion of the needle assembly with at least a portion of the at least one divider disposed in the single needle lumen and at least a portion of the at least one divider disposed in the at least one hub lumen, the at least one divider configured and arranged to physically separate the first lead from the second lead when the first lead and the second lead are received by the needle assembly.

15. The multi-lead introducer of claim 14, wherein the at least one divider is removable from the needle assembly.

16. The multi-lead introducer of claim 14, further comprising a retention assembly for maintaining the positioning of the at least one divider within the needle assembly.

17. The multi-lead introducer of claim 16, wherein at least a portion of the retention assembly is part of the single needle lumen.

18. The multi-lead introducer of claim 16, wherein at least a portion of the retention assembly is disposed on or in the hub.

19. The multi-lead introducer of claim 12, wherein at least one bend is disposed along the distal end of the single needle.

20. A multi-lead introducer for facilitating implantation of at least one electrical stimulation lead, the multi-lead introducer comprising:
a needle assembly configured and arranged to concurrently implant a plurality of leads into a patient, the needle assembly comprising
a single needle having a first end, an opposing second end, and a longitudinal length, the single needle defining a first needle lumen and a second needle lumen, each of the first needle lumen and the second needle lumen extending along the longitudinal length of the single needle from the first end to the second end, the first needle lumen configured and arranged to receive a first lead the plurality of leads, the second needle lumen configured and arranged to receive a second lead the plurality of leads,
a sharpened tip disposed along the first end of the single needle, the sharpened tip configured and arranged to pierce patient tissue, and
a hub coupled to the second end of the single needle, the hub defining at least one hub lumen that is in communication with each of the first needle lumen and the second needle lumen, the at least one hub lumen configured and arranged to concurrently receive both the first lead and the second lead.
